(19) United States
(12) Patent Application Publication
(10) Pub. No.: US 2025/0025070 A1
(43) Pub. Date: Jan. 23, 2025

(54) SWEAT SENSING DEVICE AND METHOD FOR FORMING THE SAME

(71) Applicant: AGENCY FOR SCIENCE, TECHNOLOGY AND RESEARCH, Singapore (SG)

(72) Inventors: Changyun JIANG, Singapore (SG); Wei Peng GOH, Singapore (SG); Xinting ZHENG, Singapore (SG); Yong YU, Singapore (SG); Yuxin LIU, Singapore (SG); Le YANG, Singapore (SG)

(73) Assignee: AGENCY FOR SCIENCE, TECHNOLOGY AND RESEARCH, Singapore (SG)

(21) Appl. No.: 18/713,859

(22) PCT Filed: Nov. 21, 2022
(86) PCT No.: PCT/SG2022/050846

§ 371 Date: May 28, 2024

(30) Foreign Application Priority Data
Nov. 30, 2021 (SG) 10202113328Q

(51) Int. Cl. A61B 5/145; A61B 5/00

(52) U.S. Cl. CPC A61B 5/14517; A61B 5/6804

(57) ABSTRACT

According to embodiments of the present invention, a sweat sensing device is provided. The sweat sensing device includes a continuous piece of hydrophilic paper including a first region for receiving sweat, a second region opposite the first region, and a third region therebetween; a flexible hydrophobic film having an opening; and a sensor unit. The hydrophobic film and the hydrophilic paper are arranged adjacent to each other with the opening aligned to and exposing the second region. The sensor unit is configured to facilitate a measurement based on the diffused sweat. The hydrophobic film and the hydrophilic paper are collectively folded in a stacked manner such that the sensor unit is sandwiched between the third and second regions. The hydrophilic paper is adapted for the received sweat to diffuse laterally along the hydrophilic paper. According to further embodiments, a method for forming the sweat sensing device is also provided.

### CROSS-REFERENCE TO RELATED APPLICATION

This application claims the benefit of priority of Singapore patent application No. 10202113328Q, filed 30 Nov. 2021, the content of it being hereby incorporated by reference in its entirety for all purposes.

### TECHNICAL FIELD

Various embodiments relate to a sweat sensing device and a method for forming the sweat sensing device.

### BACKGROUND

Wearable electronics for on-skin sweat sensing constantly face challenges in performing real-time, continuous and precise measurements. Sampling fresh sweat for wearable sweat sensor is a crucial part for continuous and precise on-skin sweat sensing. One key issue is sweat flow as real-time sensors need to sample fresh sweat at all times. Typically, an influx of sweat into a holding reservoir constantly replenishes “old” sweat that is removed by either passive evaporation or active pumping. Such continuous sweat flow is usually realized through a combination of conventional microfluidic channels and an active sweat flow driving mechanism, which helps to transport sweat through the sensor. However, conventional microfluidic channels usually involve high-cost fabrication processes such as photolithography or laser engraving. The integration of an active sweat flow or pumping component or mechanism complicates the sensor device and makes the wearable device bulky and costly. Both of these factors render a disposable sweat sensor impracticably expensive.

Paper has been used as sweat fluidic channels. However, such design and fabrication may be complicated. For example, a prior publication discloses a folding structure of body sweat electrochemical sensor and monitoring method where the sensor in a paper substrate is folded to form a 5-layer paper structure. Here, the folded structure has a tightly stacked arrangement of hydrophobic layers and hydrophilic layers, with an electrochemical three-electrode system integrated/embedded in one of these layers. Such a tightly stacked arrangement creates a vertical channel with interfaces interspersed within for sweat to vertically migrate through the layer-interface folded structure.

In another prior publication, a MXene double-layer paper-based electrode electrochemical sweat sensor and preparation method thereof are disclosed. Here, the sensor on the paper base material has multiple groups of double-layer structure of a three-electrode system, where MXene is used to form modified electrochemical sensor electrodes. Wax printing technology was employed to enable the paper-based material pro-hydrophobic area to form multiple microflated control channels between layers to achieve sweat collection, circulation, detection and diffusion function. This sensor is also based on a vertical channel being formed with interfaces interspersed within for sweat to vertically migrate through.

However, an easy fabrication and integration into sensors, or more preferably, multiplexed sensors for efficient sweat refreshing are still lacking.

Thus, there is a need for a novel sweat sensing device that addresses at least the problems mentioned above.

### SUMMARY

According to an embodiment, a sweat sensing device is provided. The sweat sensing device may include a continuous piece of hydrophilic paper including a first region configured to receive sweat, a second region opposite to the first region, and a third region between the first region and the second region, the continuous piece of hydrophilic paper being adapted for the received sweat to diffuse laterally along the continuous piece of hydrophilic paper from the first region to the second region via the third region; a flexible hydrophobic film having an opening, the flexible hydrophobic film and the continuous piece of hydrophilic paper being arranged adjacent to each other with the opening aligned to and exposing the second region as an outlet; and a sensor unit configured to facilitate a measurement based on the diffused sweat. The flexible hydrophobic film and the continuous piece of hydrophilic paper may be collectively folded in a stacked manner such that the sensor unit is sandwiched between the third region and the second region.

According to an embodiment, a method for forming a sweat sensing device is provided. The method may include providing a continuous piece of hydrophilic paper with a pre-determined shape of a first region for receiving sweat, a second region for evaporating the sweat, a third region for sensing the sweat; providing a flexible hydrophobic film having an opening; arranging the flexible hydrophobic film and the continuous piece of hydrophilic paper adjacent to each other with the opening aligned to and exposing the second region; providing a sensor unit over the second region; and collectively folding the continuous piece of hydrophilic paper and the flexible hydrophobic film into a stacked manner such that the sensor unit is sandwiched between the third region and the second region. The pre-determined shape may further include a first channel arranged between the first region and the third region, and a second channel arranged between the second region and the third region. The first channel and the second channel may be for the sweat to diffuse through to reach the respective regions.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1A shows a schematic view of a sweat sensing device, according to various embodiments.

FIG. 1B shows a flow chart illustrating a method for forming a sweat sensing device, according to various embodiments.

FIG. 2A shows an unassembled plan view of various parts of the multi-layer stacked paper fluidic structures of a sweat sensor, according to one example.

FIG. 2B shows an assembled plan view of FIG. 2A.

FIG. 2C shows a plan view of FIG. 2B with a sensing component placed on the multi-layer stacked paper fluidic structures.

FIG. 2D shows a plan view of FIG. 2C with one portion folded.

FIG. 2E shows a plan view of FIG. 2D with another portion folded.

FIG. 2F shows a side view of FIG. 2E.

FIG. 3 shows a side view of a two-layered paper stacked structure of sweat flow from bottom through to top, according to another example.

FIG. 4A shows a side view illustrating the sweat sensor of FIG. 2F, when in operation, according to one example.

FIG. 4B shows a side expanded view illustrating the sweat sensor of FIG. 2F, when in operation, according to another example.

FIG. 5 shows a graph of water mass change vs evaporation time of four samples as configured in FIG. 4A when operated at room temperature of 25° C. and at temperature of 37° C.

FIG. 6 shows a side view illustrating the kirigami paper fluidic with an additional evaporation pad for improved evaporation rate, in an integrated sweat sensor, according to one example.

FIG. 7 shows a graph depicting continuous monitoring of sweat biomarkers based on the integrated sweat sensor of FIG. 6, according to one example.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the phrase “substantially” may include “exactly” and a reasonable variance.

In the context of various embodiments, the term “about” as applied to a numeric value encompasses the exact value and a reasonable variance.

As used herein, the term “and/or” includes any and all combinations of one or more of the associated listed items.

As used herein, the phrase of the form of “at least one of A or B” may include A or B or both A and B. Correspondingly, the phrase of the form of “at least one of A or B or C”, or including further listed items, may include any and all combinations of one or more of the associated listed items.

As used herein, the expression “configured to” may mean “constructed to” or “arranged to”.

Various embodiments may provide a kirigami paper fluidic channel for sweat sensors. A sweat refresh method for on-skin electrochemical sweat sensors is also provided, based on the kirigami design using, for example, ultrathin cellulose paper, where the continuous sweat flow is driven by passive evaporation. The materials and fabrication process are simple and low cost, without the use of microfluidic channels and active sweat flow or pump.

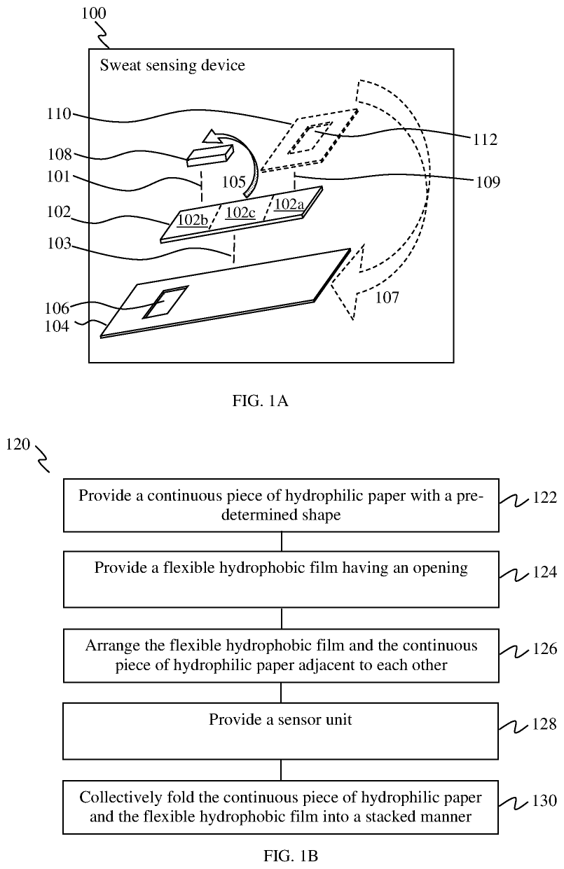

FIG. 1A shows an exploded schematic representation of a sweat sensing device 100, according to various embodiments. As seen in FIG. 1A, the sweat sensing device 100 may include a continuous piece of hydrophilic paper 102 including a first region 102a configured to receive sweat (as an inlet), a second region 102b opposite to the first region 102a, and a third region 102c between the first region 102a and the second region 102b; a flexible hydrophobic film 104 having an opening 106; and a sensor unit 108. The continuous piece of hydrophilic paper 102 may be adapted for the received sweat to diffuse laterally along the continuous piece of hydrophilic paper 102, as a sweat channel or paper channel, from the first region 102a to the second region 102b via the third region 102c. The flexible hydrophobic film 104 and the continuous piece of hydrophilic paper 102 may be arranged adjacent to each other with the opening 106 aligned to and exposing the second region 102b or a part thereof (as an outlet). This may be apparent from FIG. 1A by bringing the flexible hydrophobic film 104 and the continuous piece of hydrophilic paper 102 together along a dotted line 103. The flexible hydrophobic film 104 and the continuous piece of hydrophilic paper 102 may be collectively folded (as denoted by an arrow 105) in a stacked manner such that the sensor unit 105 is (arranged) sandwiched between the third region 102c and the second region 102b. The collective folding may be apparent from FIG. 1A by further bringing the sensor unit 108 and the continuous piece of hydrophilic paper 102 together along a dotted line 101 prior to or when folding into the stacked manner. The sensor unit 108 may be configured to facilitate a measurement (or detection) based on the diffused sweat, for example, through the third region 102c. The second region 102b may be adapted for the diffused sweat to passively evaporate via the opening 106.

In some embodiments, each of the first region 102a, the second region 102b, and the third region 102c may be of a substantially same size. In other embodiments, the first region 102a, the second region 102b, and the third region 102c may be of different sizes. The continuous piece of hydrophilic paper 102 may take on different shapes and contours to optimize the manipulation of the sweat. It should be appreciated that the continuous piece of hydrophilic paper 102 shown in FIG. 1A is only for illustrative illustration purposes.

In other words, the sweat sensing device 100 in the stacked manner may be configured to receive sweat at the first region 102a and the continuous piece of hydrophilic paper 102 may effectively form a meandering sweat channel for the sweat to flow through. The sweat may flow via capillary effect along the continuous piece of hydrophilic paper 102 from the first region 102a, then to the third region 102c and finally to the second region 102b in a meandering manner and in absence of any interfaces interspersed within the regions 102a, 102b, 102c. For example, there is no interface interspersed within the second region 102b and the third region 102c since the sensor unit 108 may be arranged planarly therebetween. Thus, even when folded in the stacked manner, the sweat is prohibited from vertically migrating across from the third region 102c to the second region 102b. Consequently, the sweat sensing device 100 advantageously has a form factor smaller than that of conventional sweat sensors. The sweat sensing device 100 utilizes passive evaporation to continuously refresh sweat at the sensing elements.

In various embodiments, the sweat sensing device 100 may further include a further or additional flexible hydrophobic film 110 having an aperture 112, the further flexible hydrophobic film 110 and the continuous piece of hydrophilic paper 102 being arranged adjacent to each other with the aperture 112 aligned to and exposing the first region 102a or a part thereof as an inlet for receiving the sweat. This may be apparent from FIG. 1A by further bringing the further flexible hydrophobic film 110 and the continuous piece of hydrophilic paper 102 together along a dotted line 109. The further flexible hydrophobic film 110 and the flexible hydrophobic film 104 may be placed on opposite surfaces of the continuous piece of hydrophilic paper 102.

Each of the flexible hydrophobic film 104 and the further flexible hydrophobic film 110 may include or may be made of polyethylene, or polyethylene terephthalate, or polyester, or polythene, or polypropylene, or polyvinyl chloride. Each of the flexible hydrophobic film 104 and the further flexible hydrophobic film 110 may be provided with adhesive on one side for adhering to the continuous piece of hydrophilic paper 102.

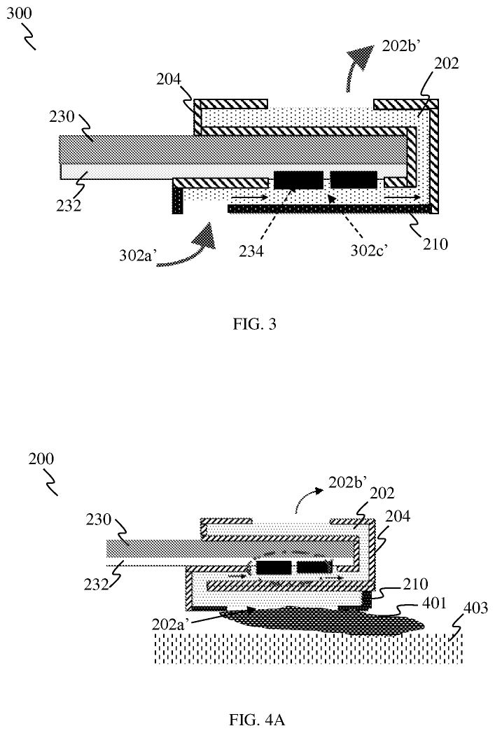

In one embodiment, the first region 102a may be extended laterally away from the third region 102c along a same plane such that the stacked manner forms a U-bended shape with the first region 102a providing a sweat collection portion arranged laterally adjacent to the third region 102c providing a sensing layer, and the second region 102b providing an evaporation layer, e.g. as seen in an example of FIG. 3. In this case, only a single inward fold may be made as denoted by the arrow 105.

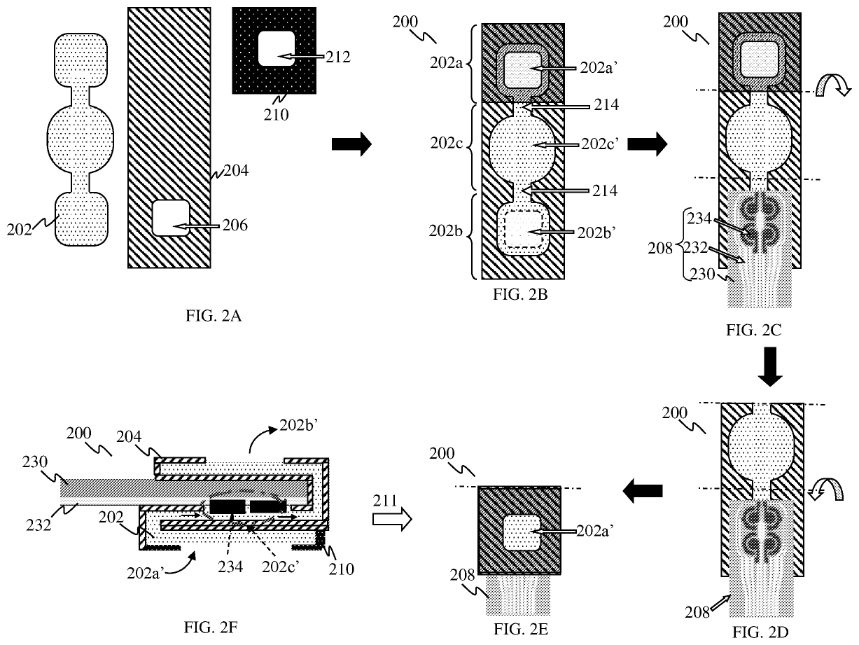

In a different embodiment, the first region 102a and the third region 102c are folded over each other with corresponding parts of the flexible hydrophobic film 104 facing each other such that the stacked manner forms a continuous zig-zag shape with the first region 102a providing a sweat collection layer, the third region 102c providing a sensing layer and the second region 102b providing an evaporation layer, e.g. as seen in an example of FIG. 2F. In the case of FIG. 2F, one inward fold may be made as denoted by the arrow 105, and another outward fold may be made as denoted by an arrow 107.

In various embodiments, the continuous piece of hydrophilic paper 102 may include a continuous piece of cellulose paper. Other paper materials may be used; however, the sensitivity and performance level may vary. The continuous piece of hydrophilic paper 102 may have a thickness ranging from about 0.01 mm to about 0.2 mm, or preferably from about 0.04 mm to about 0.06 mm. Basically, the continuous piece of hydrophilic paper 102 may be sufficiently thin, while maintaining integrity of the continuous piece of hydrophilic paper 102 even after ladened with the received sweat. The continuous piece of hydrophilic paper 102 may have a porosity larger than 50%, or preferably larger than 60%, or more preferably larger than 70%. The continuous piece of hydrophilic paper may have an average pore size larger than 20 μm, or preferably larger than 40 μm.

The sensor unit 108 may include a planar substrate; and a plurality of planar electrodes disposed on the planar substrate. The sensor unit 108 may further include a plurality of conductors electrically coupled to the plurality of electrodes, the plurality of conductors being configured to provide external electrical connections. The plurality of conductors may include silver, or copper, or gold, or other electrically conductive metals. The planar substrate may include a rigid substrate, or a flexible substrate, or a stretchable substrate. For example, the rigid substrate may include polycarbonate (PC) or polymethylmethacrylate/acrylic (PMMA). The flexible substrate may include polyimide, polyamide, polyethylene terephthalate (PET), polyethylene naphthalate (PEN), polypropylene (PP), or polyetheretherketone (PEEK). The stretchable substrate may include polydimethylsiloxane (PDMS) or styrene-ethylene-butylene-styrene (SEBS).

The plurality of planar electrodes may be a plurality of carbon electrodes. For example, the plurality of planar electrodes may include multiplexed sensing electrodes. In the stacked manner, the plurality of planar electrodes may be arranged facing towards (or adjacent to) at least one of the second region 102b or the third region 102c. In other words, the plurality of planar electrodes may be provided on a single side of the planar substrate and arranged facing towards (or adjacent to) only the third region 102c, e.g. as shown in FIGS. 2D and 2E. Alternatively, the plurality of planar electrodes provided on the single side of the planar substrate may be arranged facing towards (or adjacent to) only the second region 102b at one of its surfaces, while an opposite surface of the second region 102b may be adjacent to the opening 106. In a different example, the plurality of planar electrodes may be provided on both sides of the planar substrate and arranged facing towards (or adjacent to) both the second region 102b and the third region 102c, i.e. providing a bifacial sensor.

In various embodiments, the measurement may include a colorimetric measurement, or an electrochemical measurement. More specifically, the measurement may include an amperometric measurement, or a potentiometric measurement, or a resistive measurement, or an impedance measurement, or a transimpedance measurement.

The sweat sensing device 100 may further include an external evaporation pad placeable over the opening 106 to enhance passive evaporation of the diffused sweat.

The sweat sensing device 100 may be a wearable sweat sensing device.

FIG. 1B shows a method 120 for forming a sweat sensing device (e.g. 100), in accordance with various embodiments.

The method 120 may include the same or like elements or components as those of the sweat sensing device 100 of FIG. 1A, and as such, the same numerals are assigned and the like elements may be as described in the context of the sweat sensing device 100 of FIG. 1A, and therefore the corresponding descriptions may be omitted here.

With reference to FIG. 1B, at Step 122, a continuous piece of hydrophilic paper 102 with a pre-determined shape may be provided. The pre-determined shape may be of a first region 102a for receiving sweat, a second region 102b for evaporating the sweat, and a third region 102c for sensing the sweat. A first channel may be arranged between the first region 102a and the third region 102c to allow the first region 102a and the third region 102c to fluidic communicate with each other. A second channel may be arranged between the second region 102b and the third region 102c to allow the second region 102b and the third region 102c to fluidic communicate with each other. The first channel and the second channel may be for the sweat to diffuse through to reach the respective regions.

At Step 124, a flexible hydrophobic film 104 having an opening 106 may be provided. At Step 126, the flexible hydrophobic film 104 and the continuous piece of hydrophilic paper 102 may be arranged adjacent to each other with the opening 106 aligned to and exposing the second region 102b or a part thereof. At Step 128, a sensor unit 108 may be provided over the second region 102b such that the sensor unit 108 and the flexible hydrophobic film 104, more specifically, the opening 106, may be arranged at opposite surfaces of the continuous piece of hydrophilic paper 102. At Step 130, the continuous piece of hydrophilic paper 102 and the flexible hydrophobic film 104 may be collectively folded into a stacked manner such that the sensor unit 108 is sandwiched between the third region 102c and the second region 102b.

In various embodiments, the method 120 may further include adhering the continuous piece of hydrophilic paper 102 and the flexible hydrophobic film 104 to each other. The method 120 may further include folding the first region 102a and the third region 102c over each other, with corresponding parts of the flexible hydrophobic film facing 104 each other, such that the stacked manner forms a continuous zig-zag shape with the first region 102a providing a sweat collection layer, the third region 102c providing a sensing layer and the second region 102b providing an evaporation layer, e.g. as depicted in an example of FIG. 2F. The stacked manner forming the continuous zig-zag shape provides a non-vertical channel path, with spatial gaps in between stacked surfaces.

The method 120 may further include providing a further flexible hydrophobic film 110 having an aperture 112, the further flexible hydrophobic film 110 and the continuous piece of hydrophilic paper 102 being arranged adjacent to each other with the aperture 112 aligned to and exposing the first region 102a or a part thereof as an inlet for receiving the sweat. The further flexible hydrophobic film 110 and the flexible hydrophobic film 104 may be placed on opposite surfaces of the continuous piece of hydrophilic paper 102. Prior to providing the further flexible hydrophobic film 110, the method 120 may further include cutting the aperture 112 in the further flexible hydrophobic film 110.

In various embodiments, the method 120 may further include placing an external evaporation pad over the opening 106 to enhance passive evaporation of the diffused sweat, thereby improving sweat evaporation/refresh rate. The evaporation pad may include, for example, a piece of paper with an enlarged area.

Prior to providing the continuous piece of hydrophilic paper 102 at Step 122, the method 120 may further include cutting the continuous piece of hydrophilic paper 102 into the pre-determined shape using a stencil marker. Prior to providing the flexible hydrophobic film 104 at Step 124, the method 120 may further include cutting the opening 106 in the flexible hydrophobic film 104.

While the method described above is illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

Examples of multi-layer stacked paper fluidic structures and a kirigami fabrication process of a sweat refresh system, integrated with multiplexed sensors will be described below in more detail. However, it should be appreciated that the multi-layer stacked paper fluidic structures and the kirigami fabrication process of the sweat refresh system may also be applicable for integration with non-multiplexed sensors or any other sensors that require a fluidic feature for a constant flux of liquid/fluid delivery and removal, even in absence of specific examples described herein.

The multi-layer stacked paper fluidic structures may be described in similar context to the sweat sensing sensor 100 of FIG. 1A. The multi-layer stacked paper fluidic structures may include the same or like elements or components as those of the sweat sensing device 100 of FIG. 1A, and as such, the same ending numerals may be assigned and the like elements may be as described in the context of the sweat sensing device 100 of FIG. 1A, and therefore the corresponding descriptions may be omitted here.

The kirigami fabrication process may be described in similar context to the method 120 of FIG. 1B for forming the sweat sensing sensor 100 of FIG. 1A. The kirigami fabrication process may include the same or like elements or components as those of the method 120 of FIG. 1B, and as such, the same ending numerals may be assigned and the like elements may be as described in the context of the method 120 of FIG. 1B, and therefore the corresponding descriptions may be omitted here.

With the multi-layer stacked paper fluidic structures, a method for sweat channelling based on ultrathin and soft hydrophilic cellulose paper (e.g. Kimwipe or kitchen paper towel) for electrochemical sweat sensors may be provided. A method to realize continuous sweat refresh with high flow rate through the channel from bottom (on-skin) to top (atmosphere) by passive evaporation, for multiplexed sweat sensors may also be provided. In other words, here discloses a sweat refresh method for on-skin electrochemical sweat sensors, based on a kirigami design using ultrathin cellulose paper, where the continuous sweat-flow is driven by passive evaporation. The components including sweat collection, transportation, and evaporation are based on a continuous paper channel fabricated from a single sheet of ultrathin cellulose paper. More specifically, the paper-based sweat channel may be formed by directly cutting an ultrathin cellulose paper sheet and attaching it onto sensor electrodes. The cellulose paper sheet may be first cut to a required shape using a stencil maker. Next, the pattern may be transferred onto an adhesive polyester substrate which may be then affixed onto the sensor electrode through a series of folds to form a stacked paper fluidic so that the form factor of the sensor may be minimized, while maximizing sweat uptake/evaporation/flow rate. The eventual kirigami design may allow for sweat collection, transportation, sensing and evaporation processes through the stack, that achieves sweat refreshing. Continuous monitoring of multiple sweat biomarkers through a constant sweat flow may be realized by integrating these paper channels onto multiplexed sensing electrodes.

FIGS. 2A to 2F illustrate the kirigami-based fabrication process of the kirigami paper fluidic sweat system for an on-skin sweat sensor 200, according to one example. More specifically, FIG. 2A shows an unassembled plan view of various parts of the multi-layer stacked paper fluidic structures. FIG. 2B shows an assembled plan view of FIG. 2A. FIG. 2C shows a plan view of FIG. 2B with a sensing component 208 placed on the multi-layer stacked paper fluidic structures. FIG. 2D shows a plan view of FIG. 2C with one portion folded. FIG. 2E shows a plan view of FIG. 2D with another portion folded. FIG. 2F shows a side view of FIG. 2E as seen from directional arrow 211.

Firstly, as shown in FIG. 2A, the ultrathin cellulose paper 202 (which may be described in similar context with the continuous piece of hydrophilic paper 102 of FIG. 1A) is cut into the required shape, and two sheets of single-sided adhesive polyethylene terephthalate (PET) substrate 204, 210 with cut holes, namely an outlet hole 206 and an inlet hole 212 (which may be described in similar context with the flexible hydrophobic film 104 with the opening 106 and the further flexible hydrophobic film 110 with the aperture 112 of FIG. 1A, respectively) are also prepared. The cutting is done using a stencil maker or die cutter, and thus may be referred to as stencil cutting. Next, the cellulose paper 202, that may also be referred to as a cut paper pattern or a cut pattern, is attached to the adhesive side of the PET substrate 204, followed by attaching the other PET substrate 210 (that may also be referred to as a PET cover) onto the paper/PET substrate 202, 204 to form a PET cover/paper/PET substrate 210, 202, 204 layered structure at an upper part 202a as shown in FIG. 2B. A lower part 202b as shown in FIG. 2B formed by a paper/PET structure is further attached onto a sensing component 208 and folded to form a stacked paper fluidic structure (see FIG. 2C to 2E). A middle part 202c extends between the upper part 202a and the lower part 202b. The upper part 202a, the lower part 202b and the middle part 202c may correspond to the first region 102a, the second region 102b and the third region 102c of FIG. 1A, respectively. The sensing component 208 may be described in similar context to the sensing unit 108 of FIG. 1A. The cut pattern 202, together with the PET cover 210 and the PET substrate 204, may be fashioned with an inlet 202a′ for sweat collection, an outlet 202b′ for sweat evaporation, a stacked paper fluidic channel 214 (that may be described in similar context to the first and second channels referred in the method 120 of FIG. 1B) for sweat transportation from the inlet 202a′ to the outlet 202b′, and a sensing region 202c′ along the channel 214 for biomarker detection/sweat transportation (see FIGS. 2B and 2F). This design may be termed as a kirigami paper fluidic. Kirigami as the Japanese term suggests the involvement of folding and cutting of paper.

As shown in FIG. 2C, the paper/PET structure at the lower part 202b (i.e. the outlet 202b′) and the back of the sensing component 208, more specifically, a polyimide (PI) substrate 230 are attached to each other. Conductors 232 and carbon electrodes 234 may be disposed on the PI substrate 230. In FIG. 2C, it may be observed that the inlet 202a′ is folded backwards or outwardly with respect to the cut pattern 202 at the middle part 202c. In FIG. 2D, it may be observed that a stacked portion of the upper part 202a and the middle part 202c as described with respect to FIG. 2C is collectively folded frontwards or inwardly towards the lower part 202b, more specifically the sensing component 208. In other words, the sensing region 202c′ may be folded on or over top of the carbon electrodes 234. This way, in the stacked manner or folded structure as seen in FIGS. 2E and 2F, the sweat sensor 200 with the kirigami paper fluidic in a sandwich structure may be provided where the carbon electrodes 234 are facing towards the sensing region 202c′.

As discussed above, FIG. 2F shows a three-layer ultrathin cellulose paper 202, folded with both inlet 202a′ (for sweat intake) and outlet 202b′ (for sweat evaporation) at opposite sides of the sweat sensor 200, forming a stacked paper fluidic design. Sweat uptake occurs via the inlet 202a′, absorbed by the ultrathin cellulose paper 202. Sweat is transported through the channel 214 via capillary effects before it finally ends its passage by evaporating from the outlet 202b′. As passive evaporation takes place at the outlet 202b′, sweat is continuously replaced at the inlet 202a′. This in-built passive mechanism ensures a continuous sweat flow (reference being made to the direction arrows indicated with the cellulose paper 202 in FIG. 2F).

In an alternative example, a similar structure encompassing a two-layer ultrathin cellulose paper with a small inlet may be another possible design as shown in FIG. 3. FIG. 3 shows a side view of a two-layered paper stacked structure 300 of sweat flow from the bottom through to the top (reference being made to the direction arrows indicated with the cellulose paper 202 in FIG. 3). In this two-layered structure 300, the outlet 202b′ may be essentially the same as that of FIG. 2F, while an inlet 302a′ and a sensing region 302c′ respectively differ from the inlet 202a′ and the sensing region 202c′ of FIG. 2F. To obtain the two-layered structure 300, the folding step depicted in FIG. 2C may be omitted from the kirigami-based fabrication process and the other PET substrate (PET cover) 210 may be shaped and dimensioned to overlap or be adjacent to the third region 202c to form the sensing region 302c′. Since the inlet 302a′ and the sensor (or carbon) electrodes 234 are in fluidic communication with each other substantially along the same plane, the inlet 302a′ may only take on small dimensions due to the limited available space. Whilst this alternative design works, this may cause less efficient sweat collection as compared to the sweat sensor 200 of FIG. 2F.

Although the examples described above reflect either the three-layered cellulose paper 202 or the two-layered structure 300, it should be appreciated that sweat sensors involving other multiple-layered cellulose paper/structures may be implemented. For example, if there are more than three layers, additional intermediate stacks in the zig-zag configuration may be used to accommodate additional sensor electrodes to increase measurement types and/or capacities. This configuration advantageously provides a simple way to integrate more sensor electrodes/functions into as single device.

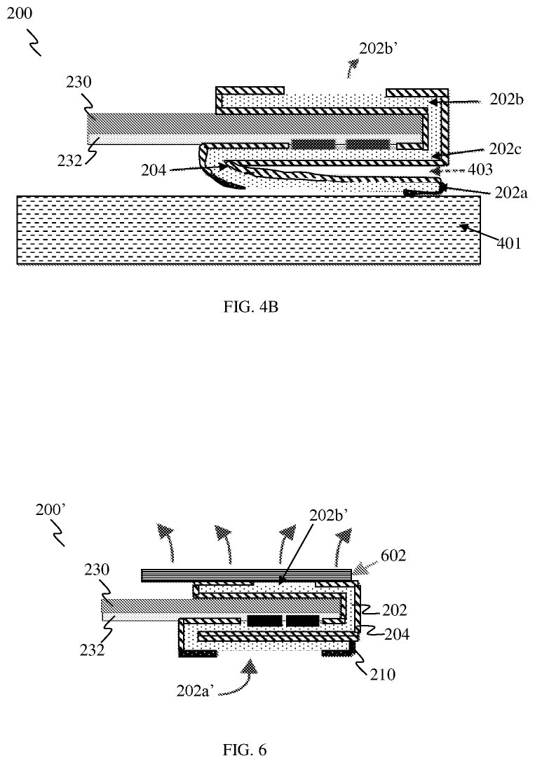

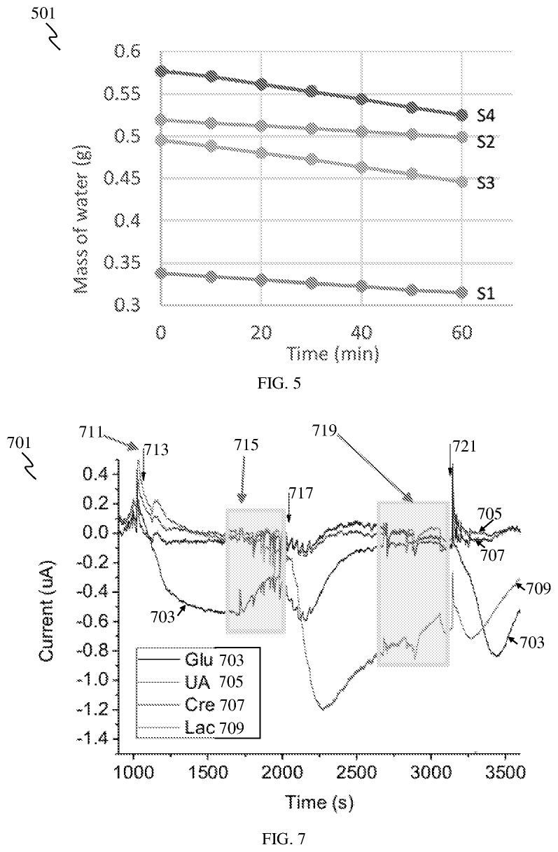

Turning back to the three-layered cellulose paper 202, FIG. 4A shows a side view illustrating the sweat sensor 200 of FIG. 2F when in operation. The sweat flow rate in the paper channel 202 may be determined by the evaporation rate through the outlet 202b′. The paper channel 202 is not a microfluidic channel, which is typically a hollow channel for fluid flow as used in conventional sweat sensors. No analyte is required to be added to work the sweat sensor 200. To measure the evaporation rate, an enclosed chamber (part of which shown as 403) injected with a known water volume 401 is attached onto the inlet 202a′. In this configuration, water may only be lost through evaporation at the outlet 202b′. Water evaporation rate may be dependent on the surface area of the outlet 202b′ and the morphology of the ultrathin cellulose paper 202 exposed at the outlet 202b′. Evaporation rate may be calculated by weighing the sweat sensor 202 of FIG. 4A at regular intervals (0, 10, 20, 40, 60 minutes). The data is summarized as a graph 501 in FIG. 5 and tabulated in Table 1.

TABLE 1
Evaporation rate of the four samples S1 to S4
as seen in FIG. 4A (Outlet area being 0.64 cm²)

| | Room temperature (25° C.) | | Temperature at 37° C. | |
|---|---|---|---|---|
| Sample ID | S1 | S2 | S3 | S4 |
| Evaporation Rate | 0.34 | 0.38 | 0.82 | 0.87 |
| (μL/min) | | | | |
| Estimated refreshing | 26 | 24 | 11 | 10 |
| time (minutes) | | | | |

With an outlet area of 0.64 cm², the evaporation rates may be determined to be 0.34 μL/min to 0.38 μL/min at room temperature (RT, 25° C.) and 0.82 μL/min to 0.87 μL/min at 37° C. (see Table 1). This may be comparable to the reported sweating rate on skin during mild exercise, for example, 0.62 μL/min/cm² and 2.58 μL/min/cm² sweating rates on arms and forehead respectively.

The thickness and porosity of the cellulose paper 202 are crucial to the sensor performance. Different cellulose papers with various thickness and porosity were tested for use as the sweat channel. It was found that thinness and good mechanical flexibility are the two most important properties of a cellulose paper to be used as the sweat channel. This ensures an intimate and conformal contact interface with the sensor electrodes (e.g. 234). A thinner paper may have a smaller channel volume at the electrode sensing area, i.e. only a small amount of sweat may be needed to flow through, hence enhancing the sweat refresh speed through the channel. Here, the channel may refer to, for example, the cut pattern 202 of FIGS. 2A to 2F and FIG. 3. High porosity with bigger pores may be advantageous for increasing the sweat flow rate in the channel. However, it should be appreciated that the cellulose paper should not be too thin nor too porous causing it to become too delicate when ladened with sweat. It was found that soft Kimwipe paper or kitchen paper towel (single layer) with thickness of about 0.04 mm to 0.06 mm were excellent candidates for use in sweat channels. Having such thinness, negligible footprint may be achieved on top of the sensor electrode. Other types of paper such as cleanroom grade polycellulose wipe (thickness of about 0.12 mm) and thick Whatman filter paper (Grade 591, thickness of about 0.18 mm) were unable to provide an accurate detection of the sweat changes. This unsuitability is mostly due to the thickness and the stiffness afforded by such papers.

FIG. 4B shows a side expanded view of the sweat sensor 200 of FIG. 2F placed on a skin 401, according to one example. As seen in FIG. 4B, gaps 403 may be provided between stacked surfaces for user comfort. Such gaps are not available in conventional sweat sensors where the sweat collection layer had to be tightly in contact with the electrode/sensing layer for the fluid connection vertically and for efficient flow, resulting in large form factor. In various embodiments and examples, spacer (or gap 403) may be allowed in between the sweat collection layer (i.e. the upper part 202a) and the electrode sensing layer (i.e. the middle part 202c), more specifically, having gaps between folded/stacked surfaces. Therefore, certain degree of freedom for movement (rotary) of the sweat collection layer may be allowed, while still maintaining the channel integrity and fluidic connection. This may provide a flexible, soft and conformal contact of the sweat collection layer with skin 401, regardless of the sensor electrode 234 and substrate (e.g. 230) used being flexible or rigid. Due to the spring-like characteristics of the folded sweat collection layer, the “gap” 403 may be auto-adjustable so that the sweat collection layer may always be in contact with the skin 401 during body motion. This design makes the contact on skin comfortable and improves the sweat sensor's 200 wearability and sweat collection efficiency. It can also be appreciated that with at least a layer of the PET substrate 204 sitting in between the sensor electrode 234 and the skin contact layer (sweat collection layer), any leakage of harmful substances (e.g. particles from the sensor electrodes) may be prevented from easily reaching the skin 401 through penetrating across the porous paper layer. This enhances the safety aspect of the sweat sensor 200, that is not catered for in conventional “vertical” stacked configuration where an entire porous paper layer with opposite surfaces directly contacts with electrode and skin, and drastically increases the chance for leaked harmful substances to contact the skin.

The sweat refresh rate in the channel is important for realizing an accurate and real-time monitoring of the sweat biomarkers. The sweat refresh time (T) may be determined by the flow rate (E, also evaporation rate here) and the volume (V) of the sensing region e.g. 202c′ (more specifically, sensor electrode e.g. 234), which may be estimated as T=V/E. For a multiplexed sensor, the total sensing region volume (V) may be relatively large. For example, the multiplexed sensor (e.g. 100, 200, 300) according to various embodiments and examples described herein may have a sensing chamber area of about 1.50 cm² and a channel height of 0.06 mm (as determined by the thickness of the cellulose paper 102, 202). Based on the evaporation rate (see Table 1), the estimated refreshing time of about 24 to 26 minutes and about 10 toll minutes at room temperature and an elevated temperature of 37°, respectively, may be still too long for accurate sweat monitoring.

To increase the sweat flow rate (also sweat evaporation rate), a larger outlet may be used. Alternatively, a paper with an enlarged area may be attached onto the outlet 202b′ of the sweat sensor 200 of FIG. 2F as an evaporation pad 602 to for an integrated sweat sensor 200′ as shown in FIG. 6 to improve the evaporation rate. Different types of paper with an area of about 2.4 cm² (rectangle) were tested for the evaporation pad. The results are summarized in Table 2.

TABLE 2
Different types of paper placed at the sweat evaporation outlet (evaporation pad)

| | | | Pore size | Evaporation Rate | | Refreshing |
|---|---|---|---|---|---|---|
| Evaporation | Area | Thickness | (μm)/ | (μL/min) | | time |
| papers | (cm²) | (mm) | porosity | RT, 25° C. | 37° C. | 37° C. |
| Kimwipe | 2.4 | 0.06 | Large | 0.83 | | |
| Whatman | 2.4 | 0.18 | Smallest | 0.91 | 1.91 | 4.7 |
| filter paper | | | (less than | | | minutes |
| (Grade 591) | | | 20 μm) | | | |
| Poly(vinyl | 2.4 | 0.50 | Small | 0.88 | | |
| alcohol) | | | | | | |
| (PVA) cloth | | | | | | |
| Melamine | 2.25 | 1.20 | Largest | 0.36 | | |
| foam | | | | | | |

It was found that a Whatman filter paper (Grade 591, thickness of about 0.18 mm) was able to attain better evaporation rates of 0.91 μL/min (RT, 25° C.) and 1.91 μL/min (37° C.). The refreshing time in the sensing channel region at 37° C. was estimated to be less than 4.7 minutes. It should be appreciated that while the example above involves evaporation pads of rectangular shape, evaporation pads of different shapes may be employed.

The sweat sensor 200′ integrated with the kirigami paper fluidic (as depicted in FIG. 6) was fabricated and characterized for continuous sweat monitoring. The measurement results are shown in a graph 701 of FIG. 7 depicting continuous monitoring of sweat biomarkers based on the sweat sensor 200′ integrated with the kirigami paper fluidic.

The kirigami paper fluidic was carefully attached onto the multiplexed sensor (i.e. to provide the integrated sweat sensor 200′) to investigate the sweat refreshing capability and the continuous monitoring of sweat biomarker levels. This integrated sweat sensor 200′ may be designed for four sweat metabolites including glucose (Glu), uric acid (UA), creatinine (Cre), and lactate (Lac) denoted by lines 703, 705, 707, 709 of FIG. 7, respectively. In this experiment, the integrated sweat sensor 200′ was first infused with sufficient amount of artificial sweat to the inlet (at point 711) until the evaporation pad 602 at the outlet 202b′ was fully wet and then, the sweat sensor 200′ was placed on a 37° C. hotplate for amperometric measurement. The artificial sweat may be applied using automatic dripping into the inlet 202a′. Upon achieving a stable baseline, at point 713, glucose solution was infused onto the inlet 202a′ (or may be referred to as inlet pad or sweat sensor inlet) of the sweat channel at around 1000 seconds at 5 μL/min continuous automatic dripping. It is observed from FIG. 7 that only line 703 representing glucose continues to show a significant drop (a response) in current as the glucose solution was being added to the sweat sensor inlet 202a′. In comparison, the other biomarkers achieved their respective baselines relatively quickly. As the amperometric signal for glucose reached a plateau, the solution dripping was manually changed to lactate solution (see point 715), and the amperometric signal for lactate dropped rather quickly as indicated by line 709 at point 717. This changing of solution was repeated one more time at point 719 as glucose solution was switched back at 3100 seconds (see point 721), and the line 703 representing glucose level quickly dropped again, indicating an increase in glucose concentration. The results of this continuous sensing experiment confirm that the kirigami paper fluidic design is useful for sweat collection and evaporation-driven refreshing. Coupling the kirigami paper fluidic with multiplexed sensors allows an integrated sweat sensor for continuous monitoring of sweat biomarker levels.

While the use of the multiplexed electrochemical sensor is demonstrated here, it should be appreciated that the sweat sensor 200, 200′, 300 does not limit the application to only electrochemical sensors. Colorimetric sensors (and any other type of sensors), which intend to measure continuous real-time sweat biomarkers, are also possible applications.

As described hereinabove, according to various embodiments and examples, the design (stacked paper fluidic) and fabrication process (kirigami) of the sweat refresh system, as well as the integration with multiplexed sensor are provided.

The kirigami paper fluidic design (e.g. the sweat sensing sensor 100, the sweat sensor 200, 200′, 300) may be designed with an inlet on the bottom (on-skin) for sweat collection, an outlet on the top (atmosphere) for sweat evaporation, and a sensing region for sweat biomarkers detection and sweat transportation from the inlet to the outlet. The three components are, for example, strung in a zig-zag multi-layered configuration so that the form factor of the sensor (more specifically, the stacked paper fluidic integrated with multiplexed sensors) may be advantageously minimized. An additional evaporation pad attached or fixed to the outlet further improves sweat evaporation and sweat refresh through the fixture. Due to the 3D nature of the design, sweat is forced to traverse in a zig-zag lateral configuration with respect to the sensing elements. This maximizes sweat uptake via the inlet, and sweat evaporation through the outlet within a limited device area. It also solely utilizes passive evaporation to continuously refresh sweat (with efficient sweat refresh rate) at the sensing elements. This may be all achieved through an inexpensive and simple fabrication process of the sweat channel, involving low cost materials as well as cutting and folding of paper, i.e. kirigami.

Any types of sensors (not limited to electrochemical applications) which require continuous real time monitoring for aqueous liquid, for instance epidermal sweat sensors in wearable electronics may be implemented.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A sweat sensing device comprising:
a continuous piece of hydrophilic paper comprising
a first region configured to receive sweat,
a second region opposite to the first region, and
a third region between the first region and the second region,
the continuous piece of hydrophilic paper being adapted for the received sweat to diffuse laterally along the continuous piece of hydrophilic paper from the first region to the second region via the third region;
a flexible hydrophobic film having an opening, the flexible hydrophobic film and the continuous piece of hydrophilic paper being arranged adjacent to each other with the opening aligned to and exposing the second region; and
a sensor unit configured to facilitate a measurement based on the diffused sweat,
wherein the flexible hydrophobic film and the continuous piece of hydrophilic paper are collectively folded in a stacked manner such that the sensor unit is sandwiched between the third region and the second region.

2. (canceled)

3. The sweat sensing device as claimed in claim 1, wherein the continuous piece of hydrophilic paper has a thickness ranging from about 0.01 mm to about 0.2 mm, or preferably from about 0.04 mm to about 0.06 mm.

4. The sweat sensing device as claimed in claim 1, wherein the continuous piece of hydrophilic paper has a porosity larger than 50%, or preferably larger than 60%, or more preferably larger than 70%.

5. The sweat sensing device as claimed in claim 1, wherein the continuous piece of hydrophilic paper has an average pore size larger than 20 μm, or preferably larger than 40 μm.

6. (canceled)

7. The sweat sensing device as claimed in claim 1, wherein the sensor unit comprises
a planar substrate; and
a plurality of planar electrodes disposed on the planar substrate.

8-9. (canceled)

10. The sweat sensing device as claimed in claim 7, wherein the planar substrate comprises a rigid substrate, or a flexible substrate, or a stretchable substrate.

11. The sweat sensing device as claimed in claim 10, wherein
the rigid substrate comprises polycarbonate or polymethylmethacrylate;
the flexible substrate comprises polyimide, polyamide, polyethylene terephthalate, polyethylene naphthalate, polypropylene, or polyetheretherketone; and
the stretchable substrate comprises polydimethylsiloxane or styrene-ethylene-butylene-styrene.

12. (canceled)

13. The sweat sensing device as claimed in claim 7, wherein the plurality of planar electrodes comprises multiplexed sensing electrodes.

14. The sweat sensing device as claimed in claim 7, wherein in the stacked manner, the plurality of planar electrodes is arranged facing towards at least one of the second region or the third region.

15-16. (canceled)

17. The sweat sensing device as claimed in claim 1, wherein the first region is extended laterally away from the third region along a same plane such that the stacked manner forms a U-bended shape with the first region providing a sweat collection portion arranged laterally adjacent to the third region providing a sensing layer, and the second region providing an evaporation layer.

18. The sweat sensing device as claimed in claim 1, wherein the first region and the third region are folded over each other with corresponding parts of the flexible hydrophobic film facing each other such that the stacked manner forms a continuous zig-zag shape with the first region providing a sweat collection layer, the third region providing a sensing layer and the second region providing an evaporation layer.

19. The sweat sensing device as claimed in claim 1, further comprising a further flexible hydrophobic film having an aperture, the further flexible hydrophobic film and the continuous piece of hydrophilic paper being arranged adjacent to each other with the aperture aligned to and exposing the first region.

20-21. (canceled)

22. The sweat sensing device as claimed in claim 1, further comprising an external evaporation pad placeable over the opening to enhance passive evaporation of the diffused sweat.

23-24. (canceled)

25. A method for forming a sweat sensing device, the method comprising:
providing a continuous piece of hydrophilic paper with a pre-determined shape of
a first region for receiving sweat,
a second region for evaporating the sweat,
a third region for sensing the sweat,
a first channel arranged between the first region and the third region, and
a second channel arranged between the second region and the third region,
wherein the first channel and the second channel are for the sweat to diffuse through to reach the respective regions;
providing a flexible hydrophobic film having an opening;
arranging the flexible hydrophobic film and the continuous piece of hydrophilic paper adjacent to each other with the opening aligned to and exposing the second region;
providing a sensor unit over the second region; and
collectively folding the continuous piece of hydrophilic paper and the flexible hydrophobic film into a stacked manner such that the sensor unit is sandwiched between the third region and the second region.

26. (canceled)

27. The method as claimed in claim 25, further comprising folding the first region and the third region over each other, with corresponding parts of the flexible hydrophobic film facing each other, such that the stacked manner forms a continuous zig-zag shape with the first region providing a sweat collection layer, the third region providing a sensing layer and the second region providing an evaporation layer.

28. The method as claimed in claim 25, further comprising providing a further flexible hydrophobic film having an aperture, the further flexible hydrophobic film and the continuous piece of hydrophilic paper being arranged adjacent to each other with the aperture aligned to and exposing the first region.

29. The method as claimed in claim 28, wherein prior to providing the further flexible hydrophobic film, the method further comprises cutting the aperture in the further flexible hydrophobic film.

30. The method as claimed in claim 25, further comprising placing an external evaporation pad over the opening to enhance passive evaporation of the diffused sweat.

31. The method as claimed in claim 25, wherein prior to providing the continuous piece of hydrophilic paper, the method further comprises cutting the continuous piece of hydrophilic paper into the pre-determined shape using a stencil marker.

32. The method as claimed in claim 25, wherein prior to providing the flexible hydrophobic film, the method further comprises cutting the opening in the flexible hydrophobic film.
